(19) United States
(12) Patent Application Publication
(10) Pub. No.: US 2012/0190653 A1
(43) Pub. Date: Jul. 26, 2012

(54) THERAPEUTIC EYE DROP COMPRISING DOXYCYCLINE AND A STABILIZER

(75) Inventors: JEFFREY P. GILBARD, Weston, MA (US); ELIZABETH GILBARD, Weston, MA (US); GREGORY FIELDSON, Morgantown, WV (US); ROBERT B. HUSON, Acton, MA (US)

(73) Assignees: Dow Pharmaceutical Sciences, Inc., Petaluma, CA (US); Advanced Vision Research, Inc., Woburn, MA (US)

(21) Appl. No.: 13/009,935

(22) Filed: Jan. 20, 2011

(51) Int. Cl. A61K 31/65; A61P 27/02

(52) U.S. Cl. 514/153

(57) ABSTRACT

The present invention provides stable aqueous doxycycline aqueous solutions suitable for pharmaceutical, especially ophthalmic, use. The doxycycline aqueous solutions have a pH ranging from 4.5-8, and contain an antioxidant and a stabilizer such as caffeine, creatine or mixtures thereof. The solutions have improved lifetimes and can be used topically.

### BACKGROUND OF THE INVENTION

This invention relates an improved aqueous-based tetracycline formulation for treating inflammatory eye diseases. The ophthalmic preparations described herein are designed for local administration in the treatment of eye surface inflammation, including meibomianitis and associated blepharitis and related dry eye diseases and disorders. Doxycycline is the preferred tetracycline.

Dry eye is one of the most prevalent forms of ocular discomfort and irritation. Estimates range up to 20 million people in the US being affected with mild dry eye, and the literature reports that as many as 3.2 million American women suffer from clinically significant dry eye. (Schaumberg et al, 2003). Dry eye can be related to external factors, such as the low humidity of air conditioned offices, winter heating, a dusty or windy outdoor environment, prolonged use of computers, or wearing of contact lenses, as well as to internal factors, such as hormonal imbalance, autoimmune disease, the presence of many widely prescribed systemic medications, anatomical changes or trauma, and aging. Chronic dry eye disease is associated with an immune-based inflammation of the lacrimal glands and the ocular surface. Symptoms result in mildly decreased quality of life at a minimum, and with increasing severity, loss of function and productivity, pain, light sensitivity, and the misery that accompanies significantly impaired vision and decreased quality of life. With the aging population in the United States and other countries of the developed world, and with increasing computer use, dry eye will become more prevalent.

It has been known that systemic administration of tetracyclines provides potent antibacterial properties throughout the body. When administered systemically, tetracycline enters into the tears and concentrates in goblet cells, around blood vessels, and on the external surface of the conjunctival epithelium. Hoeprich P D, Warshauer D M. Antimicrob Agents Chemother. 1974; 5:330-336, and Dilly P N, Mackie I A. Br J. Ophthalmol. 1985; 69:25-28. Systemic administration of tetracycline, however, has several drawbacks. For example, it often results in adverse side effects, including gastrointestinal irritation, vaginal yeast infection, sunlight sensitivity and systemic allergic reactions.

U.S. Pat. No. 6,432,934, the disclosure of which is incorporated herein by reference, described tetracycline in aqueous solutions that were used to treat ocular inflammation. These solutions were shown to be effective in reducing eyelid inflammation in an animal model for meibomitis, and also effective in increasing conjunctival goblet cell density.

However, ophthalmic preparations for topical application that contain an aqueous solution of doxycycline are typically unstable, particularly ophthalmic preparations of low osmolarity. The doxycycline breaks down and forms epimers that degrade performance. What is needed is an ophthalmic preparation comprising the tetracycline doxycycline, which is stable in an aqueous buffer, for topical application to the eye in an amount sufficient to treat an ocular disease characterized by eye surface inflammation.

### BRIEF SUMMARY OF THE INVENTION

It has now been found that stable, high potency solutions of doxycycline monohydrate can be provided by means of a novel pharmaceutical composition containing caffeine and/or creatine. The caffeine and/or creatine function as stabilizers of the doxycycline, reducing or delaying epimer formation, in an aqueous buffer of low osmolarity.

Thus, utilizing a stabilizing system that contains caffeine and/or creatine allows formation and use of a high potency aqueous solution of doxycycline.

More particularly, the novel ophthalmic pharmaceutical compositions comprising a high potency solution of doxycycline monohydrate in an aqueous buffer of low osmolarity described herein are useful in suppressing eye surface inflammation, including dry eye and meibomianitis while maintaining or restoring conjunctival mucus-containing goblet cells. Doxycycline is preferably present at a concentration of ranging from about 0.01 to 2% w/w.

The invention provides an ophthalmic preparation for topical application to the eye. The ophthalmic preparation has (a) a tetracycline, preferable doxycycline, in an amount sufficient to treat an ocular disease characterized by eye surface inflammation; (b) an aqueous buffer; (c) a stabilizer selected from the group consisting of caffeine, creatine and mixtures thereof, and (d) an antioxidant. The preparation normally has a pH ranging from 4.5-8, with a pH of 5-6 preferred and about pH 5.5 being more preferred. The caffeine is normally present at a concentration ranging from 0.05% w/w to 2.0% w/w, while the creatine, if used, is normally used at about the same concentration, ranging from 0.05 w/w to 2.0% w/w. If caffeine and creatine are used together, the total concentration of the two rarely exceeds 2.0% w/w.

Although many different antioxidants can be used, the preferred antioxidants include, but are not limited to, sodium metabisulfite, sodium thiosulfate and mixtures thereof. The sodium thiosulfate, if used, is normally present at a concentration ranging from 0.5 to 1% w/w, and the sodium metabisulfite, if present, is normally at a concentration of about 0.25% w/w.

The ophthalmic preparation may further include electrolytes. Preferred electrolytes include, but are not limited to, sodium chloride, potassium chloride, magnesium chloride hexahydrate, calcium chloride dihydrate and mixtures thereof. Preferred ranges of electrolytes are those that protect the eye and are, for example, potassium at a concentration of about 22.0 to 43.0 mM/l, bicarbonate at a concentration of about 29.0 to 50.0 mM/l, sodium at a concentration of about 130.0 to 140.0 mM/l, and chloride at a concentration of about 118.0 to 136.5 mM/l. These electrolytes are balanced to provide no significant irritation to said eye and are not toxic to the eye.

The ophthalmic preparation is preferably stable for at least 18 to 24 months at 5° C. The ophthalmic preparation may also include dibasic sodium phosphate, citric acid, and mixtures thereof and may also include a preservative. The preferred preservatives are benzalkonium chloride, methyl paraben, propyl paraben and mixtures thereof. The ophthalmic preparation may also contain sodium thiosulfate at a concentration ranging from 0.5 to 1% w/w.

The ophthalmic preparation may have an osmolarity range from 150 mOsm/Kg to 450 mOsm/Kg, preferably from 150 mOsm/Kg to 300 mOsm/Kg, or even less than 150 mOsm/Kg.

The ophthalmic preparations described herein are useful in treating eye surface disease, disorder, inflammation or dryness. The preparation as previously described is topically applied to the surface of an eye of a subject suffering from the eye surface disease, disorder, inflammation or dryness.

The ophthalmic preparation may also be used as a therapeutically effective dilution of the preparation.

### BRIEF DESCRIPTION OF THE SEVERAL VIEWS OF THE DRAWINGS

FIG. 1 illustrates the stability of six doxycycline monohydrate ophthalmic formulations (G through L) over a twelve month period at 5° C., as expressed as percent Label Claim.

FIG. 2 presents the level of epimer formation over a twelve month period at 5° C. in each of six doxycycline monohydrate ophthalmic formulations (G through L).

FIG. 3 displays the drift in pH of six doxycycline monohydrate ophthalmic formulations (G through L) over a twelve month period at 5° C.

### DETAILED DESCRIPTION OF THE INVENTION

Tetracyclines have been used for treatment of a variety of eye diseases: blepharitis, ocular rosacea, corneal inflammatory diseases and corneal infections. Normally, the treatments have been with oral dosage or some oil-based emulsions. Aqueous solutions of tetracyclines, particularly doxycycline, have had little use because of the instability of the tetracyclines in aqueous solutions.

Doxycycline, a member of the tetracycline antibiotic family and a preferred therapeutically active component of this invention, is a widely used antibiotic of high potency and having a superior half-life. It is particularly described in U.S. Pat. No. 3,200,149 under the chemical name α-6-deoxy-5-oxytetracycline.

Doxycycline is a broad spectrum antibiotic commonly used to treat a variety of infections such as chronic prostatitis, sinusitis, syphilis, pelvic inflammatory disease, acne and rosacea. Brand names include Monodox®, Periostat®, Vibramycin®, Vibra-Tabs®, Doryx®, Vibrox®, Adoxa®, and Amidox® (topical doxycycline hyclate for gum disease).

Doxycycline is also used as part of the treatment of ocular surface diseases. However, in some instances, doxycycline is used opthalmically for several reasons that are not related to its normal use as an antibiotic. By concentrating in the meibomian glands (lipid- or oil-forming glands at the edge of each eyelid), a more stable tear film is achieved. This can improve sensations of scratchiness and dryness. Altering the lipid with doxycycline may reduce free fatty acid formation by bacteria on the eyelids. Free fatty acids are similar to household detergents and may cause a burning sensation of the eye. Doxycycline blocks or inhibits some of the body's responses to infection and inflammation. Inflammation makes the eye and eyelids red and irritated. By reducing excessive inflammation, delicate tissues such as the cornea may be spared from scarring and/or destruction. Ayad A. Farjo, M D, Doxycycline Use in Cornea and External Disease, 2004 The University of Iowa, http://webeye.ophth.uiowa.edu/dept/SERVICE/CORNEA/Doxycycline/index.htm. The therapeutic value of doxycycline has also been ascribed to its ability to irreversibly inhibit corneal matrix metalloproteinase-2 (MMP-2) activity by chelating the metal ions that are catalytically and structurally essential. Smith et al. Br J. Ophthalmol. 2004; 88:619-625. Because of the difficulty in obtaining stable aqueous solutions of doxycycline, the major uses have been orally or in an oil-based preparation.

An effective concentration range for doxycycline in the solutions of this invention is generally from about 0.01 to 2% by weight of the total in the form of the free base or a pharmaceutically acceptable acid salt. The preferred form is doxycycline monohydrate, with the preferred concentration being about 0.05% by weight.

Other examples of suitable salts of doxycycline include such pharmaceutically acceptable salts as hydrochloride, hydrobromide and sulfate, including where the salt is doxycycline hydrochloride, e.g., in the form of doxycycline hyclate, which is doxycycline hydrochloride hemiethanolate hemihydrate.

The aqueous buffer for the ophthalmic preparations of the invention includes sodium chloride, potassium chloride, sodium bicarbonate, calcium chloride, magnesium chloride, sodium phosphate, borate buffer, and purified water, and mixtures thereof. Citric acid may optionally be added, e.g., for use as a phosphate citrate buffer.

Magnesium ions combine with doxycycline in solution to form magnesium-doxycycline chelates. Magnesium chloride is a convenient and preferred source of magnesium ions, but other magnesium compounds useful for the purpose of this invention include magnesium oxide, magnesium acetate and magnesium sulfate. The molar ratio of magnesium to doxycycline in these compositions is about from 1.8 to 2.2. This ratio is advisable to produce clear stable solutions.

The stability of these solutions for therapeutic administration is still further enhanced by the use of antioxidants such as sodium metabisulfite, sodium thiosulfate and mixtures thereof. Preferably, the sodium thiosulfate is present at a concentration ranging from 0.5 to 1% w/w. Preferably, the sodium metabisulfite is present at a concentration of about 0.25% w/w. Sodium metabisulfite prevents color change of the ophthalmic preparations.

Further stabilization of the ophthalmic preparations described herein is provided by the addition of a stabilizer selected from the group consisting of caffeine, creatine and mixtures thereof. Preferably caffeine is present at a concentration ranging from about 0.05% to 2%. Preferably creatine is present at a concentration ranging from about 0.05% to 2%.

Sodium carboxymethylcellulose may also be present in the ophthalmic preparations of the invention, preferably at a concentration ranging from about 0.01% to 5%, preferably at a concentration of about 0.25%. Sodium carboxymethylcellulose functions as a thickener and/or as an eye lubricant. The ophthalmic preparation may further include a preservative such as benzalkonium chloride, methyl paraben, propyl paraben and their mixtures.

The pH value is adjusted if necessary to pH 4.5 to 8. The preferred range is pH 5 to 7. The pH can be adjusted by means of an acid that is pharmaceutically acceptable, such as hydrochloric acid or by means of an organic base, such as monoethanolamine.

The compositions of this invention are readily prepared. While the order of steps is not important, normally an aqueous buffer containing electrolytes such as dibasic sodium phosphate, citric acid, sodium bicarbonate and sodium phosphate monobasic monohydrate is prepared, and the dibasic sodium phosphate and citric acid ratio is adjusted to achieve the target pH, e.g., 5.5. A tetracycline antibiotic, e.g., doxycycline, is then added, followed by addition of sodium chloride, potassium chloride, magnesium chloride hexahydrate and calcium chloride dihydrate.

The ophthalmic preparations described herein are stable over a wide temperature range and are satisfactory from a physical and chemical stability standpoint.

In contrast to topical ointments and oil-based carriers known in the art, the doxycycline composition of the present invention is formulated in an aqueous solution, preferably containing electrolytes. Suitable concentrations of doxycycline in solution include a concentration range of between about 0.01 and 2% when the solution is isotonic, hypotonic or slightly hypertonic.

The preparation preferably also includes a balance of electrolytes found in natural tear fluid required for ocular surface maintenance, function and repair. These electrolytes are present in amounts sufficient to maintain or restore conjunctival goblet cells and corneal glycogen, thereby maintaining mucus-mediated lubrication and the potential for normal healing. This enables topical application of the preparation to ocular surfaces without substantially reducing the density of conjunctival mucus-containing goblet cells or levels of corneal glycogen. Goblet cells form a critical layer of the tear film, providing the eye surface with lubrication, and playing an important role in the system that traps foreign matter that may enter the eye, and promptly removes it. Corneal glycogen is the energy source for the sliding step in corneal wound healing. Their preservation is therefore important in maintaining the health of ocular surfaces.

As used herein, the term “eye surface inflammation” includes any inflammatory disorder involving the ocular surface. The eye surface includes the eye lids, conjunctiva and cornea. Inflammation refers to white blood cell or leukocytic infiltration associated with cellular injury. Eye surface inflammatory disorders treatable by the ophthalmic preparation of the invention are typically manifested by signs and symptoms such as eye redness, or irritation. These diseases include, for example, meibomianitis, blepharitis conjunctival hyperemia, eyelid hyperemia, keratitis and ocular rosacea.

As used herein, the term “eye surface dryness” includes any ocular disorder resulting in loss of water from the tear film. Such disorders generally can be characterized by increased tear film osmolarity and decreased levels of corneal glycogen and conjunctival mucus-containing goblet cells. Eye surface dryness can result from a number of different diseases including, for example, meibomian gland dysfunction and meibomian gland orifice stenosis or closure.

Ophthalmic preparations of the invention include aqueous solutions containing one or more tetracycline compounds which are, collectively, present in an amount sufficient to treat eye surface inflammation, such as meibomianitis or eye surface redness.

As described above, the ophthalmic preparations of the invention include, in addition to doxycycline, a balance of electrolytes naturally found in tear fluid. These electrolytes principally include major amounts of sodium and chloride, and lesser amounts of potassium and bicarbonate. The preparation may also contain other naturally-occurring elements of the tear fluid, such as proteins, enzymes, lipids and metabolites as described in U.S. Pat. No. 4,911,933. Typically, the potassium is present at a concentration of about 22.0 to 43.0 mM/l, the bicarbonate is present at a concentration of about 29.0 to 50.0 mM/l, the sodium is present at a concentration of about 130.0 to 140.0 mM/l, and the chloride is present at a concentration of about 118.0 to 136.5 mM/l. The osmolarity of the resulting solution is preferably in the range of about 150 to 300 mOsm/Kg or 150 to 450 mOsm/Kg, but may also be less than 150 mOsm/Kg or greater than 450 mOsm/Kg. Water may be added or removed from the preparation to create appropriate therapeutic dilutions or concentrations.

The ophthalmic preparation can further optionally include calcium, magnesium and phosphate. To the extent present, the calcium is preferably present at a concentration of about 0.5 to 2.0 mM/l, the magnesium is preferably present at a concentration of about 0.3 to 1.1 mM/l, and the phosphate is preferably present at a concentration of about 0.8 to 2.2 mM/l.

Accordingly, the invention may provide an ophthalmic solution having an osmolarity of about 150-450 mOsm/Kg, which includes at least the following components: (a) tetracycline at a concentration of about 0.125% to 2%; (b) potassium at a concentration of about 22.0 to 43.0 mM/l; (c) bicarbonate at a concentration of about 29.0 to 50.0 mM/l; (d) sodium at a concentration of about 130.0 to 140.0 mM/l, (e) chloride at a concentration of about 118.0 to 136.5 mM/l, (f) calcium at a concentration of about 0.5 to 2.0 mM/l, (g) magnesium at a concentration of about 0.3 to 1.1 mM/l, and (e) phosphate at a concentration of about 0.8 to 2.2 mM/l. Preferred concentrations of these components range from 0.01 to 2% for tetracycline, preferably doxycycline, 23.0 to 42.0 mM/l potassium, 31.0 to 48.0 mM/l bicarbonate, 131.0 to 139.0 mM/l sodium, 124.0 to 136.0 mM/l chloride, 0.6 to 0.8 mM/l calcium, 0.5 to 0.6 mM/l magnesium, and 1.0 to 2.0 mM/l phosphate.

The preferred forms of the ophthalmic preparation are isotonic or hypotonic. However, the final osmolarity may be adjusted according to conditions present in the tear film or on the ocular surface (e.g., tear film osmolarity). For example, treatment of hypertonic tear films may make diluted preparations preferable. Conversely, preparations may be concentrated to hypertonic concentrations if therapeutically desirable. It is known that hypotonic and hypertonic eye drops are brought rapidly to isotonicity by movement of water across the eye surface (Maurice et al. (1971) Exp. Eye Res. 11:30). Thus, when treating elevated tear film osmolarity (as associated, for example, with dry eye disorders), it may be preferable to dilute the ophthalmic preparation to hypotonicity while maintaining the proportions or balance of the electrolytes disclosed herein, and adjusting the concentration of the doxycycline such that the appropriate concentration is attained after entrance of water from the solution into the eye surface.

Ophthalmic preparations of the invention can be applied to the ocular surface by various methods known in the art. For example, the preparation can be topically to the ocular surface as eye drops. The preparation can also be applied using an eye cup so that the eye is bathed. The preparation can also be applied using a continuous or near continuous infusion device for ocular surface irrigation and/or wetting and/or drug delivery. The preparation may also be applied by devices that spray solutions as required onto the surface of the eye.

The administered doxycycline ophthalmic solution is preferably administered once or twice daily. However, other dosing regimens are known to one skilled in the art. Preferred packaging is in 5 to 10 mL LDPE dropper bottle, to be stored at 5° C. to 25° C., with an osmolality of 150-180 mOsmols (hypotonic) and a viscosity of 5 to 20 centipoise, under sterile conditions, and as described in U.S. Pat. No. 6,432,934.

As previously described herein, eye surface inflammatory disorders are often associated with eye surface dryness and irritation. Animal models for such combined ocular disorders have been produced, and can be used to test the efficacy of the ophthalmic preparations provided herein. For example, a rabbit model for meibomianitis and meibomian gland dysfunction has been developed. In this animal model, meibomian gland orifice closure results in the development of inflammation around the meibomian glands (i.e., meibomianitis), inflammation in the eyelids (blepharitis), inflammation in the conjunctiva (conjunctivitis) and in an increase in tear film osmolarity and a decrease in the levels of corneal glycogen and conjunctival mucus-containing goblet cells. As demonstrated in the Examples below, ophthalmic preparations of the invention effectively treat both the eye surface inflammation (i.e., meibomianitis) and associated eye surface dryness (elevated tear film osmolarity, decreased goblet cell density and reduced corneal glycogen) exhibited by this animal model. It is recognized that results of tests using rabbits has close correlation with humans and, therefore, that the results carry over to humans.

### WORKING EXAMPLES

The Working Examples illustrate the screening process by which Applicant developed table ophthalmologic products comprising the Active Pharmaceutical Ingredient (API) of doxycycline monohydrate (Hoveon Inc, USA). In general, doxycycline monohydrate is at 0.052% w/w concentration in the formulations described in the working examples.

In a first set of experiments, the effect of different combinations of anti-oxidants and/or stabilizing agents on the stability of doxycycline monohydrate solutions at different pHs and temperatures was assessed. In a second set of experiments, each of the leading candidates were incorporated into an ophthalmologic base (TheraTears® base), and the stability of the resultant formulations was assessed.

### Example 1

### Agent Screening

Table 1 identifies thirteen stabilizing agents that were tested in doxycycline monohydrate solutions.

TABLE 1
List of Screening Agents

| | | | | | Initial |
|---|---|---|---|---|---|
| Formulation | | CAS | | IIG | Screening |
| No. | Material | Number | Function | Ophthalmic | Target |
| 1 | 5-chloro-8- | 130-16-5 | test preservative | — | Sat. |
| | hydroxyquinoline | | | | |
| 2 | antipyrine | 60-80-0 | test preservative | 0.1000% | 0.10% |
| | | 58-08-2 | | | |
| 3 | caffeine | 58-08-2 | test preservative | 2.0000% | 0.50% |
| 4 | creatine | 57-00-1 | test preservative | 0.5000% | 0.50% |
| 5 | polyvinyl | 9003-39-8 | test preservative | 0.6000% | 0.60% |
| | pyrrolidone | | | | |
| 6 | tyloxapol | 2530F02-4- | test preservative | 0.3000% | 0.30% |
| 7 | sodium bisulfite | 7631-90-5 | anti-oxidant | 0.1000% | 0.10% |
| 8 | sodium metabisulfite | 7681-57-4 | anti-oxidant | 0.2500% | 0.25% |
| 9 | sodium thiosulfate | 7772-98-7 | anti-oxidant | 5.0000% | 0.50% |
| 10 | monothioglycerol | 96-27-5 | anti-oxidant | — | 0.50% |
| 11 | Tocophersolan | 30999-06-5 | anti-oxidant | 0.5000% | 0.50% |
| | (Vitamin E TPGS) | | | | |
| 12 | edetate disodium | 6381-92-6 | chelating agent | 10.0000% | 0.50% |
| 13 | citric acid | 77-92-9 | chelating agent | 0.0500% | 0.05% |

Each of the thirteen doxycycline monohydrate formulations listed in Table 1 was analyzed for its stability in appearance (color) over time at pH 5 or pH 6, at either 5° C., 25° C. or 40° C. Doxycycline monohydrate solutions turn yellow-brown as they degrade. This is not caused by oxidation, but by hydrolysis under basic conditions or by epimerization which is acid facilitated. Epimerization is a reversible condition.

At pH 5, 5° C., all formulations were colorless over a time period of 2, 4 or 8 weeks. However, over an identical time period at pH 6, 5° C., the formulations varied in color from clear to a very slight pale yellow color. The appearance of these thirteen formulations at pH 5 and pH 6, at 25° C. and 40° C., are displayed in Table 2A and Table 2B. Formulations at pH 7 were brown to black at all temperatures.

TABLE 2A
Physical Appearance Data at pH 5

| | pH 5 | | | | | |
|---|---|---|---|---|---|---|
| | 25° C. | | | 40° C. | | |
| | 2 weeks | 4 weeks | 8 weeks | 2 weeks | 4 weeks | 8 weeks |
| 1 | Pale light amber | Slight pale yellow | Light brown | Clear light brown | Brown | Brown |
| 2 | Pale light amber | Slight pale yellow | Light brown | Clear brown | Brown | Brown |
| 3 | Pale light amber | Slight pale yellow | Light brown | Clear brown | Brown | Brown |
| 4 | Pale light amber | Slight pale yellow | Golden | Clear brown | Light brown | Light brown |
| 5 | Pale light amber | Pale yellow | Light brown | Clear brown | Brown black | Brown |
| 6 | Pale light amber | Clear yellow | Light brown | Clear dark brown | Brown black | Brown |
| 7 | Translucent yellow | slight clear yellow | Pale yellow | Light pale yellow | Pale yellow | Light yellow |
| 8 | Pale light amber | v. slight yellow | Pale yellow | Pale yellow | Pale yellow | Light yellow |
| 9 | Clear | Slight pale yellow | Cloudy | Cloudy pale yellow | Pale yellow | Light brown |
| | | | yellow | | | cloudy |
| 10 | Pale light amber | Clear yellow | Brown | Brown | Cloudy brown | Dark brown |
| 11 | Pale light amber | Clear yellow | Light brown | Brown black | Brown black | Brown |
| 12 | Pale light amber | Slight pale yellow | Golden | Pale yellow | Brown black | Light brown |
| 13 | Pale light amber | Slight pale yellow: | Light brown | Clear dark yellow | Brown black | Brown |
| 14 | Clear | Clear | Clear | Clear | Clear | Clear |

TABLE 2B
Physical Appearance Data at pH 6

| | pH 6 | | | | | |
|---|---|---|---|---|---|---|
| | 25° C. | | | 40° C. | | |
| | 2 weeks | 4 weeks | 8 weeks | 2 weeks | 4 weeks | 8 weeks |
| 1 | Light brown | Clear brown | Light brown | Brown black | Brown | Brown |
| 2 | Pale brown | Clear brown | Light brown | Brown black | Brown | Brown |
| 3 | Brown | Clear brown | Light brown | Dark brown | Brown | Brown |
| 4 | Pale brown | Clear light brown | Golden | Brown | Brown | Brown |
| 5 | Pale brown | Clear brown | Light brown | Brown black | Brown | Brown |
| 6 | Brown | Dark brown | Light brown | Brown black | Brown | Brown |
| 7 | Pale yellow | Pale yellow | Pale yellow | Pale yellow | Light brown | Brown |
| 8 | v. slight pale yellow | v. slight pale yellow | Pale yellow | Light pale yellow | Pale yellow | Light yellow |
| 9 | Cloudy pale yellow | Cloudy yellow | Cloudy yellow | Cloudy brown | Brown black | Cloud brown |
| 10 | Darker yellow | Clear dark brown | Brown | Yellow-Brown | Brown black | Dark brown |
| 11 | Brown | Clear dark brown | Light brown | Brown black | Brown black | Brown |
| 12 | Pale light brown | Clear light brown | Golden | Dark brown | Brown black | Dark brown |
| 13 | Pale light brown | Clear light brown | Light brown | Dark brown | Brown | Brown |
| 14 | Clear | Clear | Clear | Clear | Clear | Clear |

Parameters of stability other than color were also assessed in the thirteen formulations listed in Table 1. Each of the thirteen formulations was incubated for 2 weeks at 25° C. in a phosphate-citrate buffer system at pH 5, 6, and 7, containing 0.052% w/w concentration doxycycline monohydrate. The maximum % label claim (LC), that is the minimum loss of the API doxycycline monohydrate, at the end of this two week time period was then assessed in each of the thirteen solutions. These results are displayed in Table 3.

TABLE 3
Assay results t = 2 weeks at 25° C.

| Formu- | | Theoretical | pH 5 | pH 6 | pH 7 |
|---|---|---|---|---|---|
| lation | | Doxycycline | % | % | % |
| No. | Description | % w/w | LC | LC | LC |
| 1 | 5-chloro-8- | 0.05 | 96.8 | 91.3 | 29.8 |
| | hydroxyquinoline | | | | |
| 2 | Antipyrine | 0.05 | 88.2 | 85.9 | 22.9 |
| 3 | Caffeine | 0.05 | 93.6 | 85.9 | 33.8 |
| 4 | Creatine | 0.05 | 94.2> | 90 | 30.5 |
| 5 | Polyvinyl pyrrolidone | 0.05 | 76.5 | 81.2 | 21.6 |
| 6 | Tyloxapol | 0.05 | 96.2 | 82.2 | 16.5 |
| 7 | Sodium bisulfite | 0.05 | 87.1 | 76 | 66.6 |
| 8 | Sodium metabisulfite | 0.05 | 79.2 | 82.3 | 81.4 |
| 9 | Sodium thiosulfate | 0.05 | 98.4 | 92 | 75.3 |
| 10 | Monothiogylcerol | 0.05 | 93.5 | 52.9 | 46 |
| 11 | Toeophersolan | 0.05 | 94.8 | 77.5 | 20 |
| | (vitamin E TPGS) | | | | |
| 12 | EDTA disodiurn | 0.05 | 93.8 | 90.4 | 63.6 |
| 13 | Citric acid | 0.05 | 93.7 | 89.4 | 86.3 |
| 14 | Buffer | N/A | NA | NA | |

Formulation Nos. 3, 4, 9, 12 and 13 (caffeine, creatine, sodium thiosulfate; EDTA and citric acid, respectively) show the highest % LC in Table 3.

Based on their maximum % LC as described in Table 3, six doxycycline monohydrate formulations identified as Formulation Nos. 3, 4, 8, 9, 12 and 13 (caffeine, creatine, sodium metabisulfite, sodium thiosulfate, EDTA and citric acid, respectively) were further tested for their stability after 4 weeks at either pH 5 and 6. In addition to the % LC, the percentage of the 4 epimer and 6 epimer doxycycline monohydrate degradation products was measured. These results are displayed in Table 4.

TABLE 4
% LC and percentage of the 4 epimer and 6 epimer doxycycline monohydrate
degradation products after 4 weeks at pH 5 and pH 6 at 25° C.

| | | | | | | | | | | | T0-T4 |
|---|---|---|---|---|---|---|---|---|---|---|---|
| Formu- | | TO | | | T2 | | | T4 | | | Change |
| lation | | % | 4 | 6 | % | 4 | 6 | % | 4 | 6 | % |
| No. | pH 5 | LC | epimer | epimer | LC | epimer | epimer | LC | epimer | epimer | LC |
| 3 | Caffeine | 98.7 | 0.56 | ND | 93.6 | 0.29 | 0.04 | 85.7 | 8.8 | 1.03 | 13 |
| 4 | Creatine | 99.3 | 0.96 | | 94.2 | 4.09 | 0.65 | 86.1 | 8.4 | 1.3 | 13.2 |
| 8 | Sodium | 94.3 | 0.56 | | 79.2 | 5 | 0.48 | 68.8 | 9.5 | 0.98 | 25.5 |
| | metabisul- | | | | | | | | | | |
| | fite | | | | | | | | | | |
| 9 | Sodium | 104.6 | 0.53 | | 98.2 | 4.48 | 0.68 | 91.1 | 8.8 | 0.94 | 13.5 |
| | thiosulfate | | | | | | | | | | |
| 12 | EDTA | 100.1 | 0.62 | | 93.8 | 4.82 | 0.7 | 83.5 | 9.4 | 1.11 | 16.6 |
| 13 | Citric Acid | 102.3 | 0.73 | | 93.7 | 5.39 | 0.74 | 84.8 | 10 | 1.4 | 17.5 |
| | | | | | | | | | | | T0-T4 |
| | | TO | | | T2 | | | T4 | | | Change |
| | | % | 4 | 6 | % | 4 | 6 | % | 4 | 6 | % |
| | pH 6 | LC | epimer | epimer | LC | epimer | epimer | LC | epimer | epimer | LC |
| 3 | Caffeine | 98.7 | ND | ND | 85.9 | 3.8 | | 72.1 | 6 | 1.95 | 26.6 |
| 4 | Creatine | 99.3 | | | 90 | 4.3 | | 79.5 | 7.5 | 0.69 | 19.8 |
| 8 | Sodium | 94.3 | | | 82.3 | 4 | | 71.9 | 6.9 | 0.64 | 22.4 |
| | metabisul- | | | | | | | | | | |
| | fite | | | | | | | | | | |
| 9 | Sodium | 04.6 | | | 92 | 3.9 | 0.5 | 85 | 7 | 0.9 | 19.6 |
| | thiosulfate | | | | | | | | | | |
| 12 | EDTA | 100.1 | | | 90.4 | 4.3 | 0.3 | 79.9 | 7.35 | 1.77 | 20.2 |
| 13 | Citric Acid | 102.3 | | | 89.4 | 4.5 | 0.3 | 79.2 | 7.79 | 1.11 | 23.1 |

ND = Not Determined

The data in Table 4 show Formulations 3, 4, and 9, (caffeine, creatine and sodium thiosulfate, respectively) show the lowest change % LC over four weeks at pH 5.

### Example 2 Ophthalmic Formulations

Building on the stability data of the previous examples, various combinations of caffeine, sodium metabisulfite and sodium thiosulfate were assessed for their contribution to the stability of 0.052% w/w doxycycline monohydrate formulations when incorporated into an ocular pharmaceutical base having a pH of 5.5. The components of six such ophthalmic formulations are displayed in Table 5 below.

TABLE 5

| | A | B | C | D | E | F |
|---|---|---|---|---|---|---|
| Materials: | % w/w | % w/vv | % w/w | % w/w | % w/w | % w/w |
| Doxycycline | 0.052 | 0.052 | 0.052 | 0.052 | 0.052 | 0.052 |
| monohydrate (API) | | | | | | |
| Sodium CMC | 0.25 | 0.25 | 0.25 | 0.25 | 0.25 | 0.25 |
| Sodium Chloride | 0.2934 | 0.2934 | 0.2934 | 0.2934 | 0.2934 | 0.2934 |
| Potassium Chloride | 0.0966 | 0.0966 | 0.0966 | 0.0966 | 0.0966 | 0.0966 |
| Magnesium Chloride | 0.0066 | 0.0066 | 0.0066 | 0.0066 | 0.0066 | 0.0066 |
| Hexahydrate | | | | | | |
| Sodium Phosphate | 0.0074 | 0.0074 | 0.0074 | 0.0074 | 0.0074 | 0.0074 |
| monobasic monohydrate | | | | | | |
| Calcium Chloride | 0.0085 | 0.0085 | 0.0085 | 0.0085 | 0.0085 | 0.0085 |
| Dihydrate | | | | | | |
| Sodium Bicarbonate | 0.1451 | 0.1451 | 0.1451 | 0.1451 | 0.1451 | 0.1451 |
| Methyl Paraben | 0.005 | 0.005 | 0.005 | 0.005 | 0.005 | 0.005 |
| Propyl Paraben | 0.0015 | 0.0015 | 0.0015 | 0.0015 | 0.0015 | 0.0015 |
| Sodium Thiosulfate | 0.5 | 1 | 1 | 1 | 0 | 0 |
| Sodium Metabisulfite | 0.25 | 0.25 | 0.25 | 0.25 | 0 | 0 |
| Caffeine | 0 | 0 | 0.5 | 2 | 2 | 0 |
| Phosphate Citrate | qs | qs | qs | qs | qs | qs |
| buffer | | | | | | |
| Total | 100 | 100 | 100 | 100 | 100 | 100 |

Each of the ophthalmic formulations listed in Table 5 was incubated for two weeks at 30° C. The change in % LC from the initial time point was measured after two weeks. The results are displayed below in Table 6.

TABLE 6

| Formulation | | % LC | % LC | |
|---|---|---|---|---|
| Nos. | Anti Oxidant | t = 0 | t = 2 wk | ΔLC |
| A | 0.5% Na Thiosulfate | 95.45 | 76.2 | 20.25 |
| | 0.25% Na Metabilsulfite | | | |
| B | 1% Na Thiosulfate | 3.85 | 69.2 | 24.65 |
| | 0.25% Na Metabilsulfite | | | |
| C | 1% Na Thiosulfate | 96.85 | 80.1 | 16.75 |
| | 0.25% Na Metabilsulfite | | | |
| | 0.5% Caffeine | | | |
| D | 1% Na Thiosulfate | 96.15 | 82.85 | 13.3 |
| | 0.25% Na Metabilsulfite | | | |
| | 2% Caffeine | | | |
| E | 2% Caffeine | 97.1 | 77.6 | 19.5 |
| F | None | 98.65 | 72.9 | 25.75 |

The data in Table 6 indicates that ophthalmic Formulation D provides the least drop in % LC of the doxycycline monohydrate, i.e., Formulation D provides the most stabilization of Doxycycline monohydrate in this ophthalmologic base. Other observations include that caffeine alone is not enough to maintain the stability of doxycycline monohydrate as seen from Formulation E, and that sodium thiosulfate and sodium metabisulfite in combination do not provide superior protection against degradation of doxycycline monohydrate (Formulas A, B). Also, it appears that the doxycycline monohydrate is most stabilized by the combined effect of sodium thiosulfate, sodium metabisulfite and caffeine.

### Example 3

### Combinations of Oxidants and Stabilizers

Accordingly, several formulations were tested to identify leading combination of anti oxidants/stabilizers of doxycycline monohydrate in ophthalmic product prototypes. The ophthalmic formulations tested were designed to meet the narrow pH range and osmolality requirements of the commercial product profile.

An extensive set of analysis was made using each of six formulations listed in column 1 of the table below. In the first analysis the stability at 5° C. of each of these six formulations over a twelve month period is presented Table 7 below.

TABLE 7
Summary of assay and degradation data of stability through 12 months
5° C. Each formulation has 0.052% w/w Doxycycline monohydrate and 0.25%
w/w Sodium Metabisulfite in phosphate citrate buffer system pH 5.5 ± 0.5

| | T0 | Deg | % | % LC | Δ LC | Deg | % |
|---|---|---|---|---|---|---|---|
| Formula ID | % LC | products | Area | 12 mo | 12 mo | products | Area |
| G (20 mmol) | 102.5 | 4 Epimer | 0.33 | 98.9 | 3.6 | 4 Epimer | 3.84 |
| 0.5% Caffeine | | 6 Epimer | 0.47 | | | 6 Epimer | 0.44 |
| 0.5% Sodium Thiosulfate | | Total | 1.22 | | | Total | 4.96 |
| H (20 mmol) | 100.7 | 4 Epimer | 0.33 | 98.4 | 2.3 | 4 Epimer | 3.54 |
| 0.5% Caffeine | | 6 Epimer | 0.47 | | | 6 Epimer | 0.45 |
| 1% Sodium Thiosulfate | | Total | 1.30 | | | Total | 4.60 |
| I (20 mmol) | 100.0 | 4 Epimer | 0.30 | 96.1 | 3.9 | 4 Epimer | 3.50 |
| 1% Caffeine | | 6 Epimer | 0.46 | | | 6 Epimer | 0.44 |
| 0.5% Sodium Thiosulfate | | Total | 1.21 | | | Total | 4.63 |
| J (20 mmol) | 99.6 | 4 Epimer | 0.32 | 97.0 | 2.6 | 4 Epimer | 3.59 |
| 1% Caffeine | | 6 Epimer | 0.46 | | | 6 Epimer | 0.44 |
| 1% Sodium Thiosulfate | | Total | 1.25 | | | Total | 4.77 |
| K (50 mmol) | 98.5 | 4 Epimer | 0.34 | 94.4 | 4.1 | 4 Epimer | 4.39 |
| 1% Caffeine | | 6 Epimer | 0.46 | | | 6 Epimer | 0.44 |
| 1% Sodium Thiosulfate | | Total | 1.18 | | | Total | 5.91 |
| L (20 mmol/TheraTears) | 101.5 | 4 Epimer | 0.15 | 96.0 | 5.5 | 4 Epimer | 3.48 |
| 0.5% Caffeine | | 6 Epimer | 0.47 | | | 6 Epimer | 0.44 |
| 0.5% Sodium Thiosulfate | | Total | 0.95 | | | Total | 4.62 |

Deg products = Degradation Products; ALC = Delta % Label Claim

The data in Table 7 indicates that as little as 0.5% caffeine in combination with thiosulfate serves as an effective stabilizer of doxycycline monohydrate formulations.

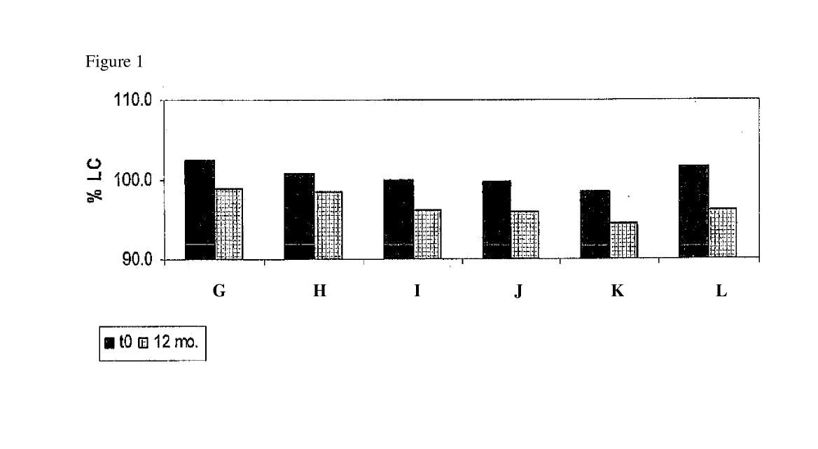

The relative stability of each of the six doxycycline monohydrate formulations G through L was assessed over a 12 month time period at 5° C. by measuring the change in percent LC at the beginning and end of this time period. The percent LC at the starting point and at the twelve month time point is presented in tabular form in FIG. 1. The data in FIG. 1 illustrates that the loss of doxycycline ranges from 2.3% to 5.5% of the label claim over a twelve month period relative to the initial t₀ value. Formulation H containing 0.5% caffeine and 0.5% sodium thiosulfate displays the least loss of doxycycline monohydrate activity.

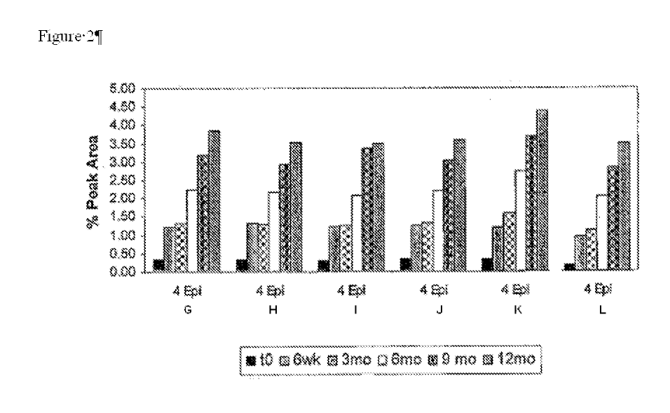

As discussed above, the development of a yellow-brown color during degradation of doxycycline monohydrate solutions is caused by epimerization which is acid facilitated. Epimerization is a reversible condition. FIG. 2 presents the level of epimer formation over a twelve month period in each of the six (G-L) doxycycline monohydrate ophthalmic formulations. Specifically, FIG. 2 illustrates that all six formulations show an increase in 4 epimer, the primary degradant of doxycycline, over a twelve month time period at 5° C. As expected, 6 epimer levels stay fairly constant from t₀ to T=12 months.

The solubility of doxycycline is influenced by the pH. The lowest pH tested is 5.5. Table 8 below displays the drift in pH of these six formulations of doxycycline monohydrate over a twelve month period at 5° C.

TABLE 8
pH Drift
5° C.

| Formula ID | tO | 2 wk | lmo. | 2 mo. | 3 mo. | 6 mo. | 9 mo | 12 mo. |
|---|---|---|---|---|---|---|---|---|
| G | 5.4 | 5.3 | 5.3 | 5.4 | 5.5 | 5.3 | 5.0 | 5.3 |
| H | 5.5 | 5.4 | 5.4 | 5.5 | 5.6 | 5.5 | 5.0 | 5.5 |
| I | 5.6 | 5.3 | 5.3 | 5.4 | 5.4 | 5.3 | 5.0 | 5.1 |
| J | 5.4 | 5.2 | 5.2 | 5.3 | 5.5 | 5.2 | 4.9 | 5.2 |
| K | 5.5 | 5.3 | 5.3 | 5.4 | 5.5 | 5.3 | 5.0 | 5.5 |
| L | 5.5 | 5.3 | 5.3 | 5.4 | 5.4 | 8.3 | 5.0 | 5.3 |

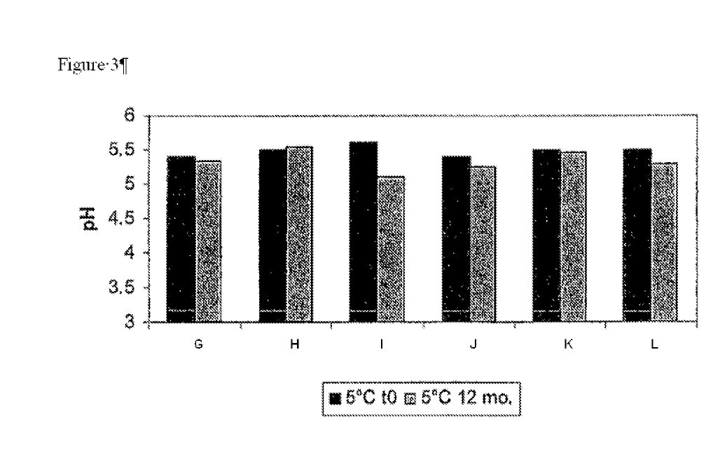

Table 8, as graphed in FIG. 3, illustrates that each of these six formulations shows very little drift in pH at 5° C. over twelve months, illustrating the stability of each of these doxycycline monohydrate formulations.

As discussed above, doxycycline monohydrate solutions turn yellow-brown as they degrade. Specifically, as each of these six formulations of doxycycline monohydrate degrade over time at 5° C., their appearance changes from a clear, colorless solution to a pale yellow color, which can be accompanied by a white precipitate. All doxycycline monohydrate formulations containing 1% caffeine stored at 5° C. present a white precipitate. Doxycycline monohydrate formulations containing 0.5% caffeine remained clear and free of any precipitate at the end of 12 months at 5° C.

TABLE 9
below displays the change in appearance of each
of these six formulations of doxycycline monohydrate
over a twelve month period at 5° C.
5° C.

| Formula ID | to | 2 wk | 1 mo. | 2 mo. | 3 mo. | 6 mo | 9 mo | 12 mo |
|---|---|---|---|---|---|---|---|---|
| G | CC | CC | CC | CC | CC | CC | CC | CC |
| H | CC | CC | CC | GC | CC | CC | CC | CC |
| I | CC | CC | CC | WM | WM | WM | WM | WM |
| J | CC | WM | WM | WM | WM | WM | WM | WM |
| K | CC | CC | CC | WM | WM | WM | WM | WM |
| L | CC | CC | CC | CC | CC | CC | CC | CC |

CC = Clear Colorless; VSPY = Very Slight Pale yellow

The data in Table 9 shows that no white precipitate is observed in doxycycline monohydrate formulations with 0.5% caffeine.

The osmolarity of each of these six doxycycline monohydrate formulations was tested at the beginning and end of a twelve month time period during which each of these six doxycycline monohydrate formulations was stored at 5° C. in glass bottles. The changes in osmolarity are displayed in Table 10 below.

TABLE 10

| | |
|---|---|
| | Osmolality |
| 5° C. | (mOsm) |

| Formula ID | t₀ | 12 mo. |
|---|---|---|
| G | 148 | 149 |
| H | 213 | 229 |
| I | 182 | 181 |
| J | 244 | 237 |
| K | 280 | 297 |
| L | 205 | 200 |

Table 10 shows that the osmolarity of each of these six doxycycline monohydrate formulations remained essentially unchanged at 5° C. at the end of twelve months.

### Example 4

### Creatine as a Stabilizer

Applicant has unexpectedly found that creatine can substitute for caffeine as a stabilizer of doxycycline monohydrate in ophthalmic formulations. A comparison of the stability during a four week time period at 25° C. of doxycycline monohydrate formulations in which creatine has been substituted for caffeine is displayed in the Tables 11A and 11B below.

TABLE 11A
Doxycycline Monohydrate Solutions 0.05% w|w, T = 4 weeks,
pH 5, 25° C.

| | | Theoretical | Determined | |
|---|---|---|---|---|
| | Description | % w/w | % w/w | % LC |
| M | Citric Acid with Dibasic Sodium | 0.05 | 0.04286 | 85.7 |
| | Phosphate and | | | |
| | caffeine | | | |
| N | Citric Acid with Dibasic Sodium | 0.05 | 0.04307 | 86.1 |
| | Phosphate and | | | |
| | creatine | | | |
| O | Citric Acid with Dibasic Sodium | 0.05 | 0.03439 | 68.5 |
| | Phosphate and | | | |
| | sodium metabisulfite | | | |
| P | Citric Acid with Dibasic Sodium | 0.05 | 0.04553 | 91.1 |
| | Phosphate and | | | |
| | sodium thiosulfate | | | |
| Q | Citric Acid with Dibasic Sodium | 0.05 | 0.04173 | 83.5 |
| | Phosphate and EDTA | | | |
| R | Citric Acid with Dibasic Sodium | 0.05 | 0.04158 | 83.2 |
| | Phosphate and citric | | | |
| | acid | | | |

TABLE 11B
Doxycycline Monohydrate Solutions 0.05% w/w, Tr: 4 weeks,
pH 6, 25° C.

| | | | | |
|---|---|---|---|---|
| M | Citric Acid with Dibasic Sodium | 0.05 | 0.03605 | 72.1 |
| | Phosphate and | | | |
| | caffeine | | | |
| N | Citric Acid with Dibasic Sodium | 0.05 | 0.03976 | 79.5 |
| | Phosphate and | | | |
| | creatine | | | |
| O | Citric Acid with Dibasic Sodium | 0.05 | 0.03594 | 71.9 |
| | Phosphate and | | | |
| | sodium metabisulfite | | | |
| P | Citric Acid with Dibasic Sodium | 0.05 | 0.04248 | 85.0 |
| | Phosphate and | | | |
| | sodium thiosulfate | | | |
| Q | Citric Acid with Dibasic Sodium | 0.05 | 0.03993 | 79.9 |
| | Phosphate and EDTA | | | |
| R | Citric Acid with Dibasic Sodium | 0.05 | 0.03961 | 79.2 |
| | Phosphate and citric | | | |
| | acid | | | |

Tables 11A and 11B show that in each formulation where caffeine has been replaced with creatine, the % LC is higher than the formulation with caffeine. Accordingly, doxycycline monohydrate ophthalmic formulations containing creatine, like caffeine, are useful in stabilizing doxycycline monohydrate formulations.

Although the invention has been described with reference to its preferred embodiments, other forms can achieve the same results. Those skilled in the art will recognize or be able to ascertain using no more than routine experimentation, numerous equivalents to the specific embodiments described herein. Such equivalents are considered to be within the scope of this invention and are encompassed by the following claims. All references and patents cited herein are hereby incorporated by reference in their entirety.

1. An ophthalmic preparation for topical application to the eye comprising (a) a tetracycline in an amount sufficient to treat an ocular disease characterized by eye surface inflammation; (b) an aqueous buffer; (c) a stabilizer selected from the group consisting of caffeine, creatine and mixtures thereof, and (d) an antioxidant, wherein said preparation has a pH ranging from 4.5-8.

2. The ophthalmic preparation of claim 1 wherein said antioxidant is selected from the group consisting of sodium metabisulfite, sodium thiosulfate and mixtures thereof.

3. The ophthalmic preparation of claim 1 further comprising an electrolyte selected from the group consisting of sodium chloride, potassium chloride, magnesium chloride hexahydrate, calcium chloride dihydrate and mixtures thereof.

4. The ophthalmic preparation of claim 1 further comprising a balance of electrolytes selected from the group consisting of potassium, chloride, bicarbonate and sodium, wherein said potassium is present at a concentration of about 22.0 to 43.0 mM/l, said bicarbonate is present at a concentration of about 29.0 to 50.0 mM/l, said sodium is present at a concentration of about 130.0 to 140.0 mM/l, and said chloride is present at a concentration of about 118.0 to 136.5 mM/l.

5. The ophthalmic preparation of claim 1, wherein said ophthalmic preparation is stable for at least 18 to 24 months at 5° C.

6. The ophthalmic preparation of claim 1, wherein said ophthalmic preparation causes no significant irritation to said eye and is not toxic to said eye.

7. The ophthalmic preparation of claim 1, wherein said pH ranges from 5-6.

8. The ophthalmic preparation of claim 1, further comprising dibasic sodium phosphate and citric acid.

9. The ophthalmic preparation of claim 1, further comprising a preservative.

10. The ophthalmic preparation of claim 9, wherein said preservative is selected from the group consisting of benzalkonium chloride, methyl paraben, propyl paraben and mixtures thereof.

11. The ophthalmic preparation of claim 1, wherein said tetracycline is doxycycline.

12. The ophthalmic preparation of claim 11, wherein said doxycycline is present at a concentration of ranging from about 0.05-0.20% w/w.

13. The ophthalmic preparation of claim 2, wherein said sodium thiosulfate is present at a concentration ranging from 0.5 to 1% w/w.

14. The ophthalmic preparation of claim 2, wherein said sodium metabisulfite is present at a concentration of 0.25% w/w.

15. The ophthalmic preparation of claim 1, wherein caffeine is present at a concentration ranging from 0.05% w/w to 2.0% w/w.

16. The ophthalmic preparation of claim 1, wherein creatine is present at a concentration ranging from 0.05% w/w to 2.0% w/w.

17. The ophthalmic preparation of claim 1, wherein said ophthalmic preparation has an osmolarity ranging from 150 mOsm/Kg to 450 mOsm/Kg.

18. The ophthalmic preparation of claim 1, wherein said ophthalmic preparation has an osmolarity ranging from 150 mOsm/Kg to 300 mOsm/Kg.

19. The ophthalmic preparation of claim 1, wherein said ophthalmic preparation has an osmolarity which is less than 150 mOsm/Kg.

20. The ophthalmic preparation of claim 1, wherein said ophthalmic preparation comprises a therapeutically effective dilution of said solution.

21. A method of treating eye surface inflammation or dryness comprising topically applying to the surface of an eye of a subject suffering from said disorder an ophthalmic preparation comprising (a) a tetracycline in an amount sufficient to treat an ocular disease characterized by eye surface inflammation; (b) an aqueous buffer; (c) a stabilizer selected from the group consisting of caffeine, creatine and mixtures thereof, and (d) an antioxidant, wherein said preparation has a pH ranging from 4.5-8.

22. The method of claim 21 wherein said antioxidant is selected from the group consisting of sodium metabisulfite, sodium thiosulfate and mixtures thereof.

23. The method of claim 21 wherein said ophthalmic preparation further comprises an electrolyte selected from the group consisting of sodium chloride, potassium chloride, magnesium chloride hexahydrate, calcium chloride dihydrate and mixtures thereof.

24. The method of claim 21 wherein said ophthalmic preparation further comprises a balance of electrolytes selected from the group consisting of potassium, chloride, bicarbonate and sodium, wherein said potassium is present at a concentration of about 22.0 to 43.0 mM/l, said bicarbonate is present at a concentration of about 29.0 to 50.0 mM/l, said sodium is present at a concentration of about 130.0 to 140.0 mM/l, and said chloride is present at a concentration of about 118.0 to 136.5 mM/l.

25. The method of claim 21, wherein said ophthalmic preparation is stable for at least 18 to 24 months at 5° C.

26. The method of claim 21, wherein said ophthalmic preparation causes no significant irritation to said eye and is not toxic to said eye.

27. The method of claim 21, wherein said preparation has a pH ranging from 5-6.

28. The method of claim 21, wherein said ophthalmic preparation further comprises dibasic sodium phosphate and citric acid.

29. The method of claim 21, wherein said ophthalmic preparation further comprises a preservative.

30. The method of claim 29, wherein said preservative is selected from the group consisting of benzalkonium chloride, methyl paraben, propyl paraben and mixtures thereof.

31. The method of claim 21, wherein said tetracycline is doxycycline.

32. The method of claim 31, wherein said doxycycline is present at a concentration of ranging from about 0.05-0.20% w/w.

33. The method of claim 22, wherein said sodium thiosulfate is present at a concentration ranging from 0.5 to 1% w/w.

34. The method of claim 22, wherein said sodium metabisulfite is present at a concentration of 0.25% w/w.

35. The method of claim 21, wherein caffeine is present at a concentration ranging from 0.05% w/w to 2.0% w/w.

36. The method of claim 21, wherein creatine is present at a concentration ranging from 0.05% w/w to 2.0% w/w.

37. The method of claim 21, wherein said ophthalmic preparation has an osmolarity ranging from 150 mOsm/Kg to 450 mOsm/Kg.

38. The method of claim 21, wherein said ophthalmic preparation has an osmolarity ranging from 150 mOsm/Kg to 300 mOsm/Kg.

39. The method of claim 21, wherein said ophthalmic preparation has an osmolarity which is less than 150 mOsm/Kg.

40. The method of claim 21, wherein said ophthalmic preparation comprises a therapeutically effective dilution of said solution.
